(19) United States
(12) Patent Application Publication
(10) Pub. No.: US 2015/0237837 A1
(43) Pub. Date: Aug. 27, 2015

(54) Non-Human Model for Wound Healing

(71) Applicant: Duke University, Durham, NC (US)

(72) Inventor: Howard Levinson, Durham, SC (US)

(21) Appl. No.: 14/408,902

(22) PCT Filed: Jun. 18, 2013
(86) PCT No.: PCT/US2013/046272

§ 371 Date: Dec. 17, 2014

Related U.S. Application Data
(60) Provisional application No. 61/660,891, filed on Jun. 18, 2012.

(51) Int. Cl. A01K 67/027

(52) U.S. Cl. CPC A01K 67/027; A01K 2267/03; A01K 2227/105; A01K 2207/30

(57) ABSTRACT

The present disclosure provides a rodent scar contracture model to study scar contractures and methods of using such a model. Such uses may include, but are not limited to, testing mechanisms or action or developing new anti-scar contracture compounds.

### BACKGROUND

Scar contracture is estimated to cost billions of dollars annually. There are over 2.4 million American burn victims annually and in major burns, the incidence of large joint scar contracture is approximately 40%. (Schneider, 2006 #937;Schneider, 2008 #936). The WHO states that “there is no doubt that the social and medical costs of burns are significant. (Organization, 2011 #3538). Economic impact of bums includes lost wages, and the costs related to deformities from burns, in terms of emotional trauma and lost skill.” (Organization, 2011 #3538). To date, there are no effective measures to prevent scar formation. This is a major unmet medical need. The lessening of scarring would improve clinical practice by reducing medical expenditures, increasing survival, and dramatically improving quality of life for millions of patients.

The present disclosure addresses these shortcomings by providing a non-human animal model to study scar contractures and methods of using such a model. Such uses may include, but are not limited to, testing mechanisms or actions or developing new anti-scar contracture compounds.

### SUMMARY OF THE INVENTION

One aspect of the present disclosure provides a method of producing a non-human animal model of scar contracture comprising of: (a) producing a full-thickness dermal wound on the skin of the animal: (b) excising the wound; and (c) securing a skin graft over the wound thereby producing a non-human animal model of scar contracture.

In one embodiment, the method further comprises the step of applying a bolster to the skin graft. In other embodiments, the method further comprises the step of removing the bolster after a period of at least two days.

In some embodiments, the full-thickness dermal wound is selected from a group consisting of a thermal injury, an excisional injury, or a combinations thereof.

In other embodiments, the non-human animal is selected from the group consisting of a rodent, cat, dog or combination thereof. In some embodiments, the non-human animal is a rodent. In certain embodiments, the non-human animal is a mouse.

Another aspect of the present disclosure provides a non-human animal model which bears an excised wound and skin graft and which is produced by the method provided herein.

Another aspect of the present disclosure provides a method of determining whether an agent has anti-scarring contracture properties for the prevention or treatment of scar contracture comprising of administering to a non-human animal having a skin grafted full-thickness dermal wound according to the present disclosure an agent of interest to the scar, measuring graft contraction in the presence of the agent, wherein an agent that reduces graft contraction and/or enables the graft to heal more like normal tissue is considered to have anti-scarring contracture properties.

In one embodiment, the wound tissue is assayed methods selected from the group consisting of histology, immunohistology, qRTPCT, Western Blot, and combinations thereof.

### BRIEF DESCRIPTION OF THE DRAWINGS

The foregoing aspects and other features of the invention are explained in the following description, taken in connection with the accompanying drawings, wherein:

FIG. 1 is a schematic detailing the method according to one embodiment of the present disclosure. FIG. 1A shows a flow diagram of a method of producing a non-human animal model of scar contracture. FIG. 1B is a graph showing results of skin contraction in one embodiment of the model.

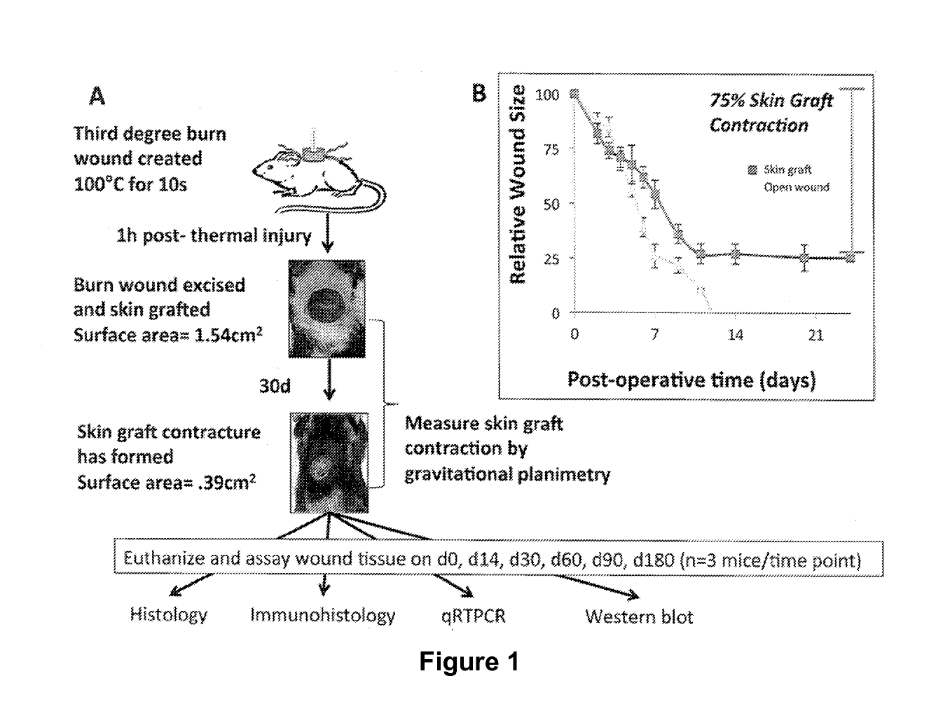

FIG. 2 are photographs demonstrating that the non-animal model of the present disclosure is analogous to the human condition. The rodent burn contracture model is analogus to the human condition with faster graft contraction; human skin grafts contract 35% by six weeks, the rodent skin graft contracts 75% by 4 weeks and the rodent excisional wound contracts 95%.

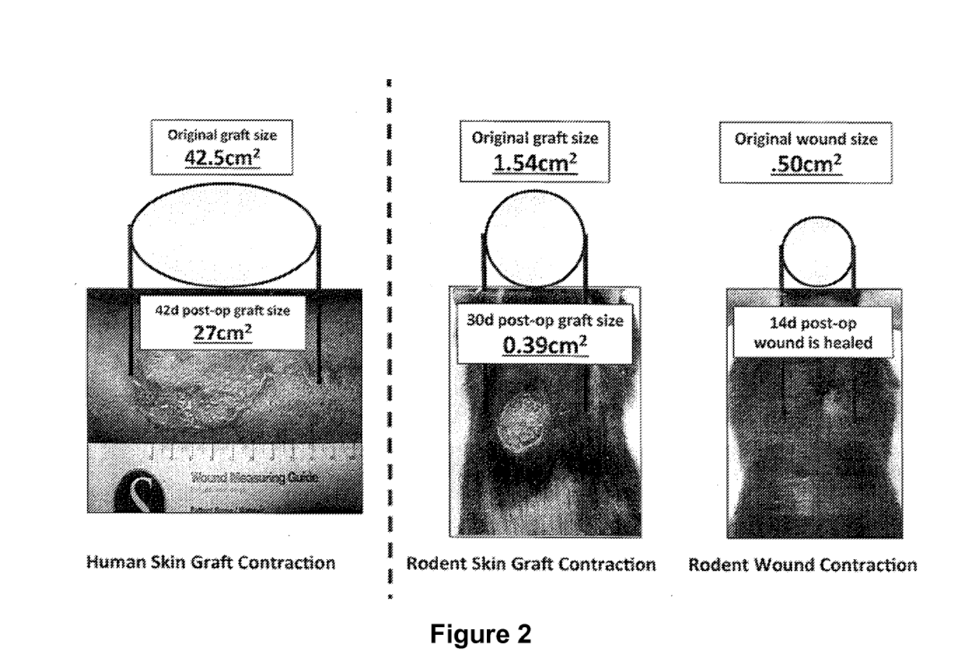

### DETAILED DESCRIPTION OF THE INVENTION

For the purposes of promoting an understanding of the principles of the present disclosure, reference will now he made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Unless specifically defined, all technical and scientific terms used herein have the same meaning as commonly understood by a skilled artisan in enzymology, biochemistry, cellular biology, molecular biology, cosmetics, and the medical sciences (e.g., dermatology, etc.). All methods and materials similar or equivalent to those described herein can be used in the practice or testing of the present disclosure, with suitable methods and materials being described herein.

Articles “a” and “an”' are used herein to refer to one or to more than one (i.e. at least one) of the grammatical object of the article. By way of example, “an element” means at least one element and can include more than one element.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of elements that do not materially affect the basic and novel or functional characteristic(s) of that embodiment of the invention.

The term “consisting of” refers to methods and respective components thereof as described herein, which are exclusive of any element not recited in that description of the embodiment.

As used herein, the term “administer” or “administering” refers to providing, contacting, and/or delivery of an agent (e.g., an agent being tested for anti-scarring properties) by any appropriate route to achieve the desired effect. These compounds may be administered to a subject in numerous ways including, but not limited to, orally, ocularly, nasally, intravenously, topically, as aerosols, suppository, etc. and may be used in combination.

As used herein, the term “therapeutically effective” refers to a dosage of an agent (e.g., an agent being tested for anti-scarring properties) that is effective for eliciting a desired effect. This term as used herein may also refer to an amount effective at bringing about a desired in vivo effect in an animal, mammal, or human, such as reducing graft contraction and/or enabling the graft to heal more like normal tissue. A therapeutically effective amount may be administered in one or more administrations, applications or dosages and is not intended to be limited to a particular formulation, combination or administration route. It is within the scope of the present disclosure that the agent may be administered at various times during the course of graft healing. The times of administration and dosages used will depend on several factors, such as the goal of treatment (e.g., treating v. preventing), condition of the subject, etc. and can be readily determined by one skilled in the art.

In certain embodiments, the agent may he in the form of a pharmaceutical composition. As used herein, the term “pharmaceutical composition” refers to the combination of an agent (i.e., for example, an agent being tested for anti-scarring properties) with a carrier, inert or active, making the composition especially suitable for diagnostic or therapeutic use in vivo or ex vivo. A “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975)). Exemplary pharmaceutical compositions comprise ALDHbr cells and a pharmaceutical carrier.

The present disclosure concerns provision of a non-human animal model representing human wound contractures (which also may be referred to as a scar contracture model) and a method for producing such a non-human animal contracture model. The non-human animal model according to the present disclosure is useful for evaluating over time the biological processes associated with scarring, as well as the anti-scarring effects of biologics, cosmetics and quasi-drugs, among other products.

Suitable animals for the present invention are any non-human mammals, including but not limited to, rodents, such as mice, rats, hamsters, gerbils and the like, rabbits, cats and dogs. In some embodiments, the animal is a rodent. In certain embodiments, the rodent is a mouse.

The goal in wound healing is an epithelialized wound. Certain wounds, such as third degree burn injuries, cause full thickness dermal wounds. These wounds can heal either by second intention (i.e., being left open to contract and epithelialize) or by skin grafting. Skin grafting expedites epithelialization. Contractures result from the persistent contraction after a wound has epithelialized. Herein, a novel non-human animal model to study contractures is provided.

The method of the present disclosure enables, without taking a long time, provision of a non-human animal model exhibiting scar/wound contractures consistently, and use of such a model enables more precise elucidation of the mechanism of scar contracture and more accurate evaluation of anti-scarring substances.

According to the present disclosure, the non-human animal scar contraction model is produced as follows: (1) creating a full-thickness dermal injury on the non-human animal (e.g., rodent) skin; (2) excising the wounded tissue; (3) covering the open wound with a skin graft; and optionally (4) applying a bolster. After the desired period of time (e.g., 2-6 days), the bolster is removed and the graft has “taken”.

The injuries applied can vary in size and/or shape, and may be the result any type of insult, such as a thermal injury, excisional injury or the like.

In certain embodiments, the skin graft according to step (3) is harvested from another rodent (e.g., mouse) by taking the skin graft from the dorsum of the rodents ears, leaving the cartilage intact. The skin graft may be placed immediately upon the excision or the wound may be allowed to heal for a period of days and then the skin graft placed.

Until grafting to an animal, the skin graft may be preserved in a suitable culture medium, such as DMEM supplemented with L-glutamine and an antibiotic/antimycotic (Invitrogen, CA), under sparse conditions while the temperature is maintained at 2-4° C.

Skin transplantation to an animal may be carried out in accordance with a methods known in the art (see, e.g., Demarchez M., Hartmann D J, Herbage D., and Ville G. Dev Biol. 1987; 121(1): 119-29). For example, the following method may be used: Under anesthesia with isofluorane/oxygen or Nembutal, a 2×2 to 3×3 cm² wound incision is made in the dorsal skin of the animal. Before the incision was made, the skin is preferably shaved. Subsequently, a skin graft having the same size is transplanted to the incision, and then sutured with a Nylon suture (10-20 stitches). Upon completion of suturing, analgesic treatment is preferably carried out by adding sensorcaine to the border between the skin graft and mouse skin. Until the mice have recovered from anesthesia, they are kept in a 37° C. incubator.

The graft is secured to the open wound by any of a variety of methods to secure the graft to the wound. Typically, a bolster is applied. After several days, the bolster is removed and the skin graft has “taken”. The graft can then be studied longitudinally over months.

Skin graft contraction, or shrinkage of skin graft, may be measured in any one of a variety of ways on a periodic basis. The graft can be assayed by a variety of techniques including but not limited to biochemical, molecular, histologic and immunhistologic assays. A biologic that reduces graft contraction and enables the graft to heal more like normal tissue is considered to have anti-scar contracture properties. Thus, the scar contracture model of the present disclosure is useful for the elucidation of the mechanism of scar/wound contracture and the evaluation of biologics, cosmetics, drugs and the like for anti-scarring contracture properties.

The present invention also relates to methods of determining the determination of whether an agent has anti-scarring properties. An agent is considered to have anti-scar contraction properties (also referred to herein as “anti-scarring properties) if it educes graft contraction and/or enables the graft to heal more like normal tissue. This method involves providing a non-human animal having a skin graft as provided in the present disclosure. The method also involves administering an agent to the incision and determining whether the agent exhibits anti-scarring properties. As used herein, the term “agent” may include drugs, prodrugs, biologics, cosmetics and the like which may exhibit anti-scarring properties. The term “agent” as used herein is also meant to encompass one or more agents, and any combination thereof.

In this aspect of the present invention, a suitable non-human animal model is one that has been made according to the method described herein.

The administration of a suitable agent to the incision is preferably dermal, i.e., the agent to be tested is topically applied to the wound. However, the agent can also be administered orally, parenterally, subcutaneously, intravenously, intramuscularly, intraperitoneally, by intranasal instillation, or by application to mucous membranes, such as that of the nose, throat, and bronchial tubes.

The agent may be administered at any time after the graft has been placed on the animal. For example, the agent may be administered once, twice or three times daily after the graft has been placed on the animal. In other embodiments, the administration of the agent may begin after the graft has taken. In yet other embodiments, the agent may be given once, twice or three times every other day. Such times and frequency of administration will depend on the agent being tested and can be readily determined by one skilled in the art at the time.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

The Examples that follow are illustrative of specific embodiments of the invention, and various uses thereof They set forth for explanatory purposes only, and are not to be taken as limiting the invention.

### Example 1

### Generation of Rodent Burn Sear Conturture Model

Female C57BL/6 mice, 10-12 weeks-old, weighing 18 to 23 g (Jackson Laboratories, Bar Harbor, Me.), were used as wild type (WT) mice throughout the study. Female hemizygous C57BL/6-Tg(CAG-EGFP)131Osb/LeySopJ carriers (green fuorescent protein;GFP) and non-control littermates, 10-12 weeks-old, weighing 18 to 23 g (Jackson Laboratories, Bar Harbor, Me.) were used for WT-GFP and GFP-WT studies. All mice were monitored for signs of toxicity including significant weight change, grooming, irritability and respiratory rate. The mice were housed under protocols approved by the institutional Animal Care and Use Committee (IACUC) of Duke University.

Mice were anesthetized using gas anesthesia (O₂, 2L/min, isoflurane, 2%). The recipient mouse was anesthetized and the back of the mouse was shaved with metallic clippers. The back was then sterilized using alcohol. The mouse was placed in a restraining tube. A metal brass rod 8 mm in diameter, weighing 65 g, was heated to 100° C. in water and placed on the back of the mouse for 1s to produce a third degree burn. The burn was left for 3 days and a 14 mm diameter circle over the burn site was excised for recipient skin grafting.

The donor mouse was anesthetized. Its ears were cleansed with alcohol and cut at the base with curved surgical scissors. The dorsal skin surface of the ear was carefully separated from the cartilaginous ventral surface using sharp fine tipped scissors, and the cartilaginous surface was discarded. Fenestrations were created in the dorsal ear skin using a #10 scalpel. Two donor skin our were laid over an excised 14min diameter burn wound. The edges of the grafts and the edges of the skin were approximated and sutured with interrupted stitches (5 stitches for each ear and 2 stitches between the 2 ears) using 6-0 silk suture. The skin grafts were then secured with a padded bolster. The bolster was removed on post-operative day 3.

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1.-10. (canceled)

11. A method of producing a non-human animal model of scar contracture comprising: (a) producing a full-thickness dermal wound on the skin of the animal; (b) excising the wound; and (c) securing a skin graft over the wound thereby producing a non-human animal model of scar contracture.

12. The method according to claim 11, wherein the full-thickness dermal wound is selected from a group consisting of a thermal injury, an excisional injury, or combinations thereof.

13. The method according to claim 11, wherein the non-human animal is selected from the group consisting of a rodent, cat, or dog.

14. The method according to claim 13, wherein the non-human animal is a rodent.

15. The method according to claim 14, wherein the non-human animal is a mouse.

16. The method according to claim 11, further comprising the step of applying a bolster to the skin graft.

17. The method according to claim 16, wherein the full-thickness dermal wound is selected from a group consisting of a thermal injury, an excisional injury, or combinations thereof.

18. The method according to claim 16, wherein the non-human animal is selected from the group consisting of a rodent, cat, or dog.

19. The method according to claim 18, wherein the non-human animal is a rodent.

20. The method according to claim 19, wherein the non-human animal is a mouse.

21. The method according to claim 16, further comprising the step of removing the bolster after a period of at least two days.

22. The method according to claim 21, wherein the full-thickness dermal wound is selected from a group consisting of a thermal injury, an excisional injury, or combinations thereof.

23. The method according to claim 21, wherein the non-human animal is selected from the group consisting of a rodent, cat, or dog.

24. The method according to claim 23, wherein the non-human animal is a rodent.

25. The method according to claim 24, wherein the non-human animal is a mouse.

26. A non-human animal model which bears an excised wound and skin graft and which is produced by the method according to claim 11.

27. A method of determining whether an agent has anti-scarring contracture properties for the prevention or treatment of scar contracture comprising (a) administering to a non-human animal having a skin grafted full-thickness dermal wound according to claim 11 an agent of interest to the scar, (b) measuring graft contraction in the presence of the agent, wherein an agent that reduces graft contraction and/or enables the graft to heal more like normal tissue is considered to have anti-scarring contracture properties.

28. The method according to claim 27, wherein wound tissue is assayed by methods selected from the group consisting of histology, immunohistology, qRTPCT, Western Blot, and combinations thereof.
